(19) United States
(12) Patent Application Publication
(10) Pub. No.: US 2022/0081397 A1
(43) Pub. Date: Mar. 17, 2022

(54) HYDROGEN-BONDED ORGANIC FRAMEWORK FOR SEPARATING ALKENES FROM ALKANES

(71) Applicant: THE BOARD OF REGENTS OF THE UNIVERSITY OF TEXAS SYSTEM, Austin, TX (US)

(72) Inventors: Banglin CHEN, San Antonio, TX (US); Zhangjing ZHANG, San Antonio, TX (US); Shengchang XIANG, San Antonio, TX (US)

(73) Assignee: THE BOARD OF REGENTS OF THE UNIVERSITY OF TEXAS SYSTEM, Austin, TX (US)

(21) Appl. No.: 17/478,425

(22) Filed: Sep. 17, 2021

Related U.S. Application Data
(60) Provisional application No. 63/079,855, filed on Sep. 17, 2020.

(51) Int. Cl. C07D 209/82; C07D 219/04; B01J 20/22; B01J 20/28; B01J 20/282; C07C 7/12

(52) U.S. Cl. CPC C07D 209/82; C07D 219/04; B01J 20/22; C07C 7/12; B01J 20/2808; B01J 20/282; B01J 20/28061

(57) ABSTRACT

In some aspects, the present disclosure provides one or more compounds of the formula:

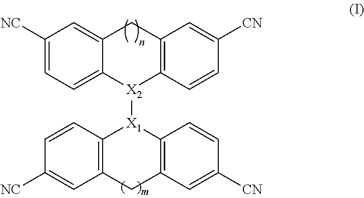

The compounds maybe used to form one or more organic frameworks that may be used in the separation of two or more molecules from each other. In some embodiments, the molecules are ethylene and ethane. In some embodiments, the organic frameworks may be used to separate one or more of these molecules with high selectivity.

### REFERENCE TO RELATED APPLICATIONS

This application claims the benefit of priority to U.S. Provisional Application No. 63/079,855, filed on Sep. 17, 2020, the entire content of which is hereby incorporated by reference.

### BACKGROUND

### I. Field

The present disclosure relates generally to the fields of chemistry and materials science. More particularly, it concerns organic frameworks, methods of preparation thereof, compositions thereof and methods of use thereof, including separating gas molecules such as ethylene and ethane.

### II. Description of Related Art

Separation is one of the very important processes in chemical industry (Sholl and Lively, 2016; Liu, 2016). Cryogenic distillation, the well-established and reliable separation technology, costs about 10-15% of the world's energy consumption, it is thus highly demanding to developing alternative and energy-efficient separation technologies (Chu et al., 2017; Kuznicki et al., 2001). Among diverse technologies, the adsorption-based ones on the porous adsorbents through pressure swing adsorption (PSA) and thermal swing adsorption (Kuznicki et al., 2001) are very promising. In fact, some adsorbents have already been implemented in industrial separations. For example, the molecular gate adsorbent ETS-4 for the industrial scale separation of natural gas separation (Kuznicki et al., 2001; Kuznicki et al., 2003).

Porous metal-organic frameworks (MOFs) (Furukawa et al., 2013), covalent-organic frameworks (COFs) (Huang et al., 2016; Li et al., 2014) and hydrogen bonded-organic frameworks (HOFs) (He et al., 2011; Liu et al., 2019), as novel adsorbents, have been developed for gas separation and purification (Liao et al., 2017; Chen et al., 2019; Zhang et al., 2020). The rational pore tuning and straightforward pore functionalization have enabled MOFs to be superior adsorbents to others for a lot of different gas separations. (Vaidhyanathan et al., 2010; Bloch et al., 2012; Yang et al., 2014; Cui et al., 2016; Yoon et al., 2016; Wang et al., 2018) Although extensive research has been pursued to target porous MOFs for gas separations, rare examples of MOFs with high sieving effects have been realized, particularly for the hydrocarbon separations (Wang et al., 2018; Liu et al., 2018; Cadiau et al., 2016; Bavykina and Gascon, 2018). This is because most hydrocarbons to be separated have very close molecular dimensions. For example, ethylene has the dimension of about 3.28×4.18×4.84 Å³, while ethane has the dimension of about 3.81×4.08×4.82 Å³. Furthermore, even some porous MOFs might presumably have high sieving separations from the structure point of view (pore/aperture sizes), it is still very challenging and difficult to fulfill high sieving separations because of the flexible nature of MOFs in which the pore spaces will be gradually enlarged under slightly higher pressures to entrap the larger hydrocarbons, generating the co-adsorption. This is clearly demonstrated in the developed UTSA-200 for propyne (C₃H₄)/propylene (C₃H₆) (Li et al., 2018), which significantly affects the separation performance and purity of the separating product. Until now, the MOF material with the best sieving separation performance for hydrocarbon separations is UTSA-280 for ethylene/ethane separation (Liu et al., 2018). The high sieving separation is attributed to the rigidity of UTSA-280 constrained by the squarates, which has almost completely blocked the entrance of the ethane molecules into the pores of UTSA-280. Given the fact that most organic linkers within MOFs will lead to flexible MOFs through their rotation and distortion under different stimulus such as temperatures and pressures (Krause et al., 2016; Gu et al., 2019; Yang et al., 2019), it is still a daunting challenge to realize rigid MOFs for high sieving separations of hydrocarbons.

Given the usefulness of materials that can effectively separate industrial feedstocks, such as ethylene from ethane, in order to obtain purer ethylene and/or purer ethane, materials that can achieve these separations are of great importance, including methods and processes to fabricate these MOFs, for example, in a large-scale, environmentally friendly, and/or economically manner.

### SUMMARY

In some aspects, the present disclosure provides organic frameworks formed through a hydrogen bond network. The present disclosure provides compounds of the formula:

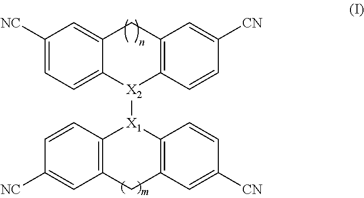

wherein:

X₁ and X₂ are each independently CH or N; and

m and n are each independently 0, 1, 2, or 3.

In some embodiments, the compounds are further defined as:

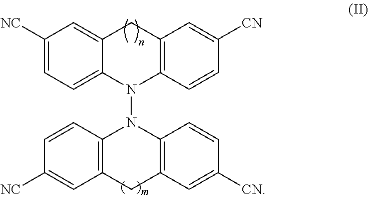

In some embodiments, the compounds are further defined as:

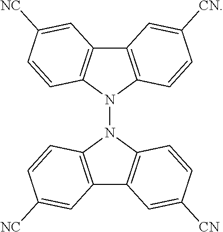 [SMILES: N#Cc1ccc2c(c1)c1cc(C#N)ccc1n2-n1c2ccc(C#N)cc2c2cc(C#N)ccc21]

In still another aspect, the present disclosure provides frameworks comprising a repeating unit of a compound described herein. In some embodiments, the repeating units are joined by non-covalent interactions. In some embodiments, the non-covalent interactions are between the nitrogen atom of the cyano and the adjacent hydrogen atom on the ring system. In some embodiments, the frameworks contain a plurality of pores from about 3 Å to about 5 Å such as from about 3.5 Å to about 4.5 Å. In some embodiments, the frameworks have a surface area from about 300 m²/g to about 500 m²/g as measured by the Brunauer-Emmett-Teller method such as from about 375 m²/g to about 425 m²/g. In some embodiments, the frameworks further comprise an alkene such as ethylene.

In still another aspect, the present disclosure provides methods of separating a C2-C6 alkene from a mixture comprising contacting the mixture with the framework described herein. In some embodiments, the mixture comprises a mixture of C1-C6 alkane and C2-C₆ alkene. In some embodiments, the alkane is ethane. In some embodiments, the alkene is ethylene. In some embodiments, the methods are carried out at a temperature below 100° C. such as from about 40° C. to about 80° C. In some embodiments, the frameworks have a selectivity for alkene over alkane of greater than 10. In some embodiments, the methods are carried out at a pressure from about 0.25 bar to about 5 bar.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present disclosure. The invention may be better understood by reference to one of these drawings in combination with the detailed description of specific embodiments presented herein.

FIGS. 1A-1B: Tuning gate-pressures for sieving separation. (FIG. 1A) Equation representing the sorption equilibrium. (FIG. 1B) Variation in the gas adsorption isotherms of a flexible-robust adsorbent with temperatures (from T_{low} to T_{high}), for smaller gas a and larger gas b, respectively.

FIGS. 2A-2E: Crystal structure of HOF-FJU-1. (FIG. 2A) Synthetic scheme of the organic ligand for the construction of HOF-FJU-1. (FIG. 2B) Intermolecular hydrogen bonding connections between cyano groups. (FIG. 2C) Three-fold interpenetrated framework with highlight in dia topology. (FIG. 2D) Framework with pore channels along the crystallographic [100] direction. (FIG. 2E) Schematic diagram of the size sieving separation for C₂H₄ and C₂H₆ molecules.

FIG. 3: The powder X-ray diffraction patterns for HOF-FJU-1.

FIG. 4: Diamondoid cage in a single-fold network.

FIG. 5: Three-fold interpenetrated diamond networks in HOF-FJU-1.

FIGS. 6A-6B: (FIG. 6A) Offset π-π stacking interactions along an axis. (FIG. 6B) Multiple intermolecular interactions in the HOF-FJU-1.

FIGS. 7A-7D: (FIG. 7A-B) Illustration of the pore channel in HOF-FJU-1 viewed along the crystallographic b-axis and a-axis; (FIG. 7C) and (FIG. 7D) Illustration of cross section of the pore aperture and cavity.

FIG. 8: The TGA curves for HOF-FJU-1 under nitrogen atmosphere with heating rate of 10° C. min⁻¹.

FIG. 9: Variable-temperature powder X-ray diffraction patterns of HOF-FJU-1.

FIG. 10: N₂ sorption isotherms of HOF-FJU-1a at 77 K.

FIG. 11: CO₂ sorption isotherms of HOF-FJU-1a at 196 K

FIG. 12: BET (FIG. 12—top) and Langmuir (FIG. 12—bottom) surface areas of HOF-FJU-1a obtained from the N₂ adsorption isotherm at 77 K.

FIGS. 13A-13L: C₂H₄ and C₂H₆ sorption and separation in HOF-FJU-1a. (FIG. 13A, FIG. 13B and FIG. 13C) Gas adsorption isotherms for ethylene and ethane in HOF-FJU-1a at 298, 318 and 333 K, respectively. Filled and open symbols represent adsorption and desorption, respectively. (FIGS. 13D, 13E and 13F) Breakthrough curves for C₂H₄/C₂H₆ mixture (50:50, v/v) in a fixed bed packed with HOF-FJU-1a at 298, 318 and 333 K, respectively. (FIG. 13G, FIG. 13H and FIG. 13I) Concentration curve of the desorbed C₂H₄ from HOF-FJU-1a during the regeneration process. Desorption was carried out by applying vacuum at 298, 318 and 333 K, respectively. (FIG. 13J) A multiple cycling test of C₂H₄/C₂H₆ (50:50, v/v) mixtures, and (FIG. 13K) breakthrough curves of HOF-FJU-1a for H₂/C₃H₆/CH₄/C₃H₈/C₂H₆/C₂H₄ mixture (5:5:5:5:40:40 v/v/v/v/v/v) at 333 K and 1 bar. (FIG. 13L) The purities of the generated C₂H₄ from HOF-FJU-1 during the regeneration processes of the fixed bed at different temperatures.

FIG. 14: Experimental data (sphere) and fitting curve (solid line) of C₂H₄ and C₂H₆ adsorption isotherms of HOF-FJU-1a at 318 and 333 K. The fitting curves are obtained by the virial-type expression.

FIG. 15: The derivation of the isosteric heat of adsorption (Qₛₜ) uses the virial equation of C₂H₄ and in HOF-FJU-1a.

FIGS. 16A-16D: The single-site Langmuir-Freundlich fitting for adsorption of C₂H₄ (FIG. 16A, FIG. 16B) and C₂H₆ (FIG. 16C, FIG. 16D) on HOF-FJU-1a at 273 and 298 K.

FIGS. 17A-17D: The single-site Langmuir-Freundlich equations fitting for adsorption of C₂H₄ (FIG. 17A, FIG. 17B) and C₂H₆ (FIG. 17C, FIG. 17D) on HOF-FJU-1a at 318 and 333 K.

FIG. 18: IAST selectivity of HOF-FJU-1a for C₂H₄:C₂H₆ (50:50, v/v) at 273, 296, 318 and 333 K.

FIGS. 19A-19C: The calculation for captured amount of C₂H₄ during the breakthrough process in HOF-FJU-1a. During the duration before breakthrough point the amount of C₂H₄ at different temperatures. (FIG. 19A) Q_{298K}=qt=0.028 mmol/min×37 min=1.036 mmol corresponding to 0.942 mmol/g. The max amount of C₂H₄ during 0-40 min Q_{max 298K}=

$$q {\int }_{0}^{∞} [ {C}_{i}^{0} - {C}_{i} ( t ) ] dt = 1.067$$

mmol corresponding to 0.97 mmol/g. (FIG. 19B) Q_{318K}=qt=0.028 mmol/min×22 min=0.616 mmol corresponding to 0.56 mmol/g. The max amount of C₂H₄ during 0-27.5 min

$${Q}_{max 381 K} = q {\int }_{0}^{∞} [ {C}_{i}^{0} - {C}_{i} ( t ) ] dt = 0.677$$

mmol corresponding to 0.6154 mmol/g. (FIG. 19C) Q_{333K}=qt=0.028 mmol/min×14 min=0.392 mmol corresponding to 0.356 mmol/g. The max amount of C₂H₄ during 0-19 min

$${Q}_{max 333 K} = q {\int }_{0}^{∞} [ {C}_{i}^{0} - {C}_{i} ( t ) ] dt = 0.448$$

mmol corresponding to 0.407 mmol/g.

FIGS. 20A-20B: Different Fourier maps with electron density peaks before (FIG. 20A) and after (FIG. 20B) loading into HOF-FJU-1.

FIGS. 21A-21C: Single-crystal structure of HOF-FJU-1·0.75 C₂H₄. (FIG. 21A) Top views of the packing diagram of the C₂H₄-loaded structure. The framework and pore surface are shown in gray and pale gold. (FIG. 21B) and (FIG. 21C) Preferential binding sites for C₂H₄ molecules and their close contacts with the framework. The white and green, red, and blue spheres represent H and C atoms of C₂H₄.

FIG. 22: Powder X-ray diffraction patterns of HOF-FJU-1 under variable pH conditions.

FIG. 23: Powder X-ray diffraction patterns of HOF-FJU-1 under different solvent conditions.

FIGS. 24A-24C: HOF-FJU-1 recrystallization process. Photographs of (FIG. 24A) HOF-FJU-1 soaked in 1 mL of DMF without dissolving. (FIG. 24B) The clear solution after 10 minutes of heating. (FIG. 24C) After remaining at room temperature for 1 day, colorless rod-like crystals were obtained (54%).

### DESCRIPTION OF ILLUSTRATIVE EMBODIMENTS

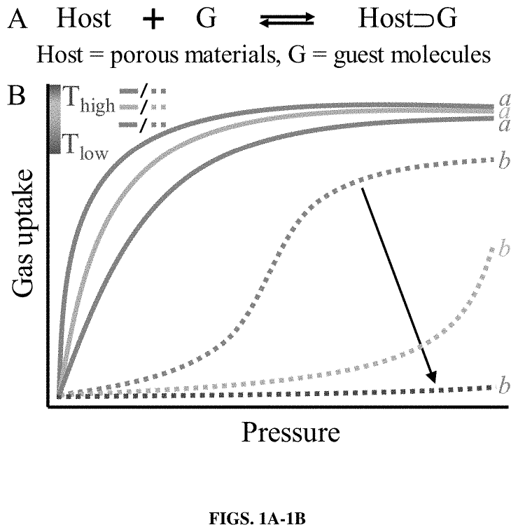

Gate pressure adsorption phenomena have been well established back in 2003 (Kituara et al., 2003). As shown in FIG. 1, adsorption/desorption is an equilibrium physical and exothermic process, which means that increasing the adsorption temperature will favor the equilibrium to the left (less gas uptakes) while increasing the gas pressure (concentration of gas) will favor the equilibrium to the right (more gas uptakes) (FIG. 1A). A porous flexible-robust material with the possible sieving pore sizes may fully make use of the robust pore spaces to take up large amount of the smaller hydrocarbons, while the flexible pore spaces will control the co-adsorption of the larger hydrocarbon molecules through the different temperatures (and correspondingly different gate pressures), as shown in FIG. 1B. This strategy has not been realized yet in any porous materials for gas separations. This disclosure provides an example of microporous flexible-robust materials with tunable gate pressures at different temperatures for the high sieving separation of ethylene from ethane at 60° C. is reported. The framework, termed as HOF-FJU-1, is a microporous hydrogen bonded-organic framework self-assembled from organic linker 3,3′6,6′-tetracyano-9,9′-bicarbazole. The recovered ethylene reached purity of 99.1% at 60° C., as clearly established by the experimental breakthrough.

### I. Organic Frameworks

In some aspects, the present disclosure provides compounds that maybe used for preparing an organic framework. The compounds include a compound of the formula:

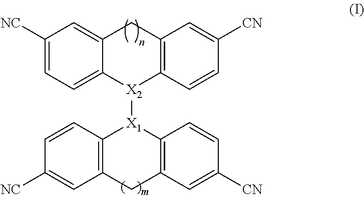

wherein: X₁ and X₂ are each independently CH or N; and m and n are each independently 0, 1, 2, or 3.

In some embodiments, X₁ is CH₂. In other embodiments, X₁ is N. In some embodiments, X₂ is CH₂. In other embodiments, X₂ is N. In some embodiments, m is 0 or 1. In some embodiments, m is 1. In some embodiments, n is 0 or 1. In some embodiments, n is 0.

In some embodiments, the compounds are further defined as:

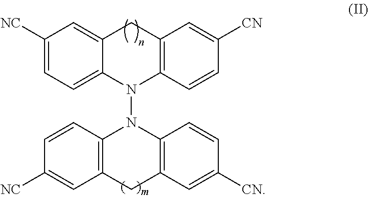

In some embodiments, the compounds are further defined as:

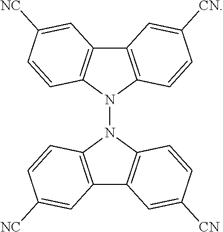 [SMILES: N#Cc1ccc2c(c1)c1cc(C#N)ccc1n2-n1c2ccc(C#N)cc2c2cc(C#N)ccc21]

In another aspect, the present disclosure provides frameworks comprising a repeating unit of a compound described herein. In some embodiments, the repeating units are joined by non-covalent interactions. In some embodiments, the non-covalent interactions are between the nitrogen atom of the cyano and the adjacent hydrogen atom on the ring system. In some embodiments, the framework contains a plurality of pores from about 3 Å to about 5 Å such as from about 3.5 Å to about 4.5 Å. In some embodiments, framework has a surface area from about 300 m²/g to about 500 m²/g as measured by the Brunauer-Emmett-Teller method such as from about 375 m²/g to about 425 m²/g. In some embodiments, the frameworks further comprises an alkene such as ethylene.

The compounds of the present invention are shown, for example, above, in the summary of the invention section, and in the claims below. The OF's discussed herein may be made using the synthetic methods outlined in the Examples section. These methods can be further modified and optimized using the principles and techniques of chemistry as applied by a person skilled in the art. In addition, the synthetic methods may be further modified and optimized for preparative, pilot- or large-scale production, either batch or continuous, using the principles and techniques of process chemistry as applied by a person skilled in the art. Such principles and techniques are taught, for example, in Anderson, Practical Process Research & Development—A Guide for Organic Chemists (2012), which is incorporated by reference herein.

### II. Methods of Chemical Separation Using OFs

In another aspect, the present disclosure provides OFs which may be used to remove one type of molecules from a mixture. In one aspect, the present disclosure provides methods of separating two or more compounds using an organic framework as described herein, wherein the OF comprises a repeating unit of the formula:

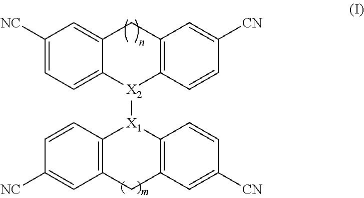

or a hydrate thereof, wherein the method comprises:
(A) combining the organic framework with a mixture comprising a first compound and a second compound; and
(B) separating the first compound from the second compound within the organic framework.

In some embodiments, X₁ is CH₂. In other embodiments, X₁ is N. In some embodiments, X₂ is CH₂. In other embodiments, X₂ is N. In some embodiments, m is 0 or 1. In some embodiments, m is 1. In some embodiments, n is 0 or 1. In some embodiments, n is 0.

In some embodiments, the framework comprises a repeating unit of the formula:

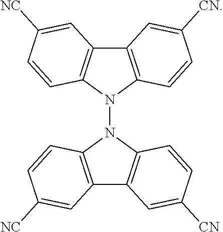

In some embodiments, the first compound or the second compound is a gas molecule. In some of these embodiments, both the first and second compounds are gas molecules. In some embodiments, the first compound is an alkene_{(C≤8)} such as ethylene. In other embodiments, the first compound is an alkyne_{(C≤8)} such as ethyne. Therefore, the methods of the present disclosure may facilitate almost complete removal of ethane from ethylene. In still other embodiments, the first compound is CO₂. In some embodiments, the second compound is an alkane_{(C≤8)} such as ethane or methane. In other embodiments, the second compound is N₂.

In some embodiments, the mixture comprises from about 1:999 to about 1:1 of the first compound to the second compound. In other embodiments, the mixture comprises from about 1:999 to about 1:1 of the second compound to the first compound. In some embodiments, the mixture comprises about 1:99 of the first compound to the second compound. In some embodiments, the separation is carried out at a pressure from about 0.1 bar to about 10 bar such as at a pressure of about 1 bar.

In some embodiments, the organic framework is adhered to a fixed bed surface. In some embodiments, the separation is carried out in an absorber packed with the metal-organic framework. In some embodiments, the separation is carried out at a temperature from about 0° C. to about 75° C. such as at about room temperature.

In still another aspect, the present disclosure provides a method of separating ethylene from a mixture of ethane and ethylene comprising exposing the mixture to a organic framework as described herein.

### III. Definitions

“organic frameworks” (OFs) are framework materials, typically three-dimensional, self-assembled by the coordination of functional groups on organic linkers exhibiting porosity, typically established by gas adsorption. The OFs discussed and disclosed herein are at times simply identified by their repeat unit as defined below without brackets or the subscript n.

The term “unit cell” is basic and least volume consuming repeating structure of a solid. The unit cell is described by its angles between the edges (α, β, γ) and the length of these edges (a, b, c). As a result, the unit cell is the simplest way to describe a single crystal X-ray diffraction pattern.

A “repeat unit” is the simplest structural entity of certain materials, for example, frameworks and/or polymers. In the case of a polymer chain, repeat units are linked together successively along the chain, like the beads of a necklace. For example, in polyethylene, —[—CH₂CH₂—]ₙ—, the repeat unit is —CH₂CH₂—. The subscript “n” denotes the degree of polymerization, that is, the number of repeat units linked together. When the value for “n” is left undefined, it simply designates repetition of the formula within the brackets as well as the polymeric and/or framework nature of the material. The concept of a repeat unit applies equally to where the connectivity between the repeat units extends into three dimensions, such as in organic frameworks, cross-linked polymers, thermosetting polymers, etc. Note that for OFs the repeat unit may also be shown without the subscript n.

“Pores” or “micropores” in the context of organic frameworks are defined as open space within the OFs; pores become available, when the OF is activated for the storage of gas molecules. Activation can be achieved by heating, e.g., to remove solvent molecules.

“Multimodal size distribution” is defined as pore size distribution in three dimensions.

“Multidentate organic linker” is defined as ligand having several binding sites for the coordination of one or more functional groups.

In addition, atoms making up the compounds of the present invention are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C. Additionally, it is contemplated that one or more of the metal atoms may be replaced by another isotope of that metal. In some embodiments, the calcium atoms can be ⁴⁰Ca, ⁴²Ca, ⁴³Ca, ⁴⁴Ca, ⁴⁶Ca, or ⁴⁸Ca. Similarly, it is contemplated that one or more carbon atom(s) of a compound of the present invention may be replaced by a silicon atom(s). Furthermore, it is contemplated that one or more oxygen atom(s) of a compound of the present invention may be replaced by a sulfur or selenium atom(s).

Any undefined valency on a carbon atom of a structure shown in this application implicitly represents a hydrogen atom bonded to the atom.

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result.

The term “hydrate” when used as a modifier to a compound means that the compound has less than one (e.g., hemihydrate), one (e.g., monohydrate), or more than one (e.g., dihydrate) water molecules associated with each compound molecule, such as in solid forms of the compound.

The term “saturated” when referring to an atom means that the atom is connected to other atoms only by means of single bonds.

The above definitions supersede any conflicting definition in any of the reference that is incorporated herein by reference. The fact that certain terms are defined, however, should not be considered as indicative that any term that is undefined is indefinite. Rather, all terms used are believed to describe the invention in terms such that one of ordinary skill can appreciate the scope and practice the present invention.

### III. Examples

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1: Synthesis and Characterization of a Flexible-Robust Hydrogen-Bonded Organic Framework

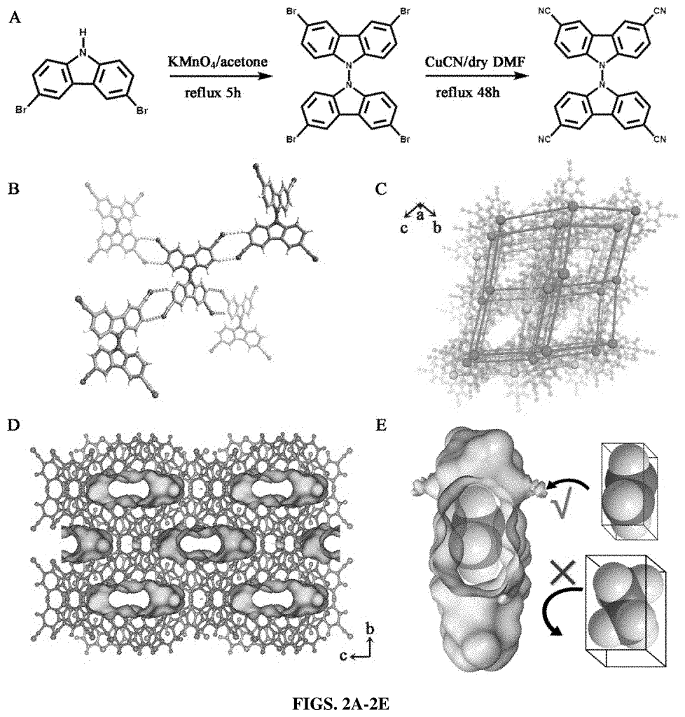

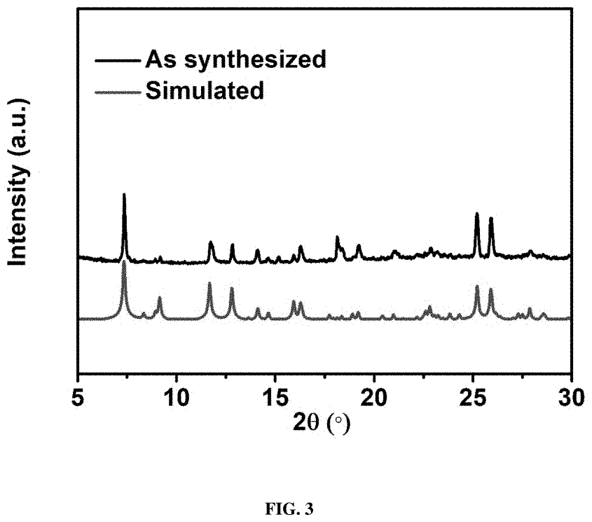

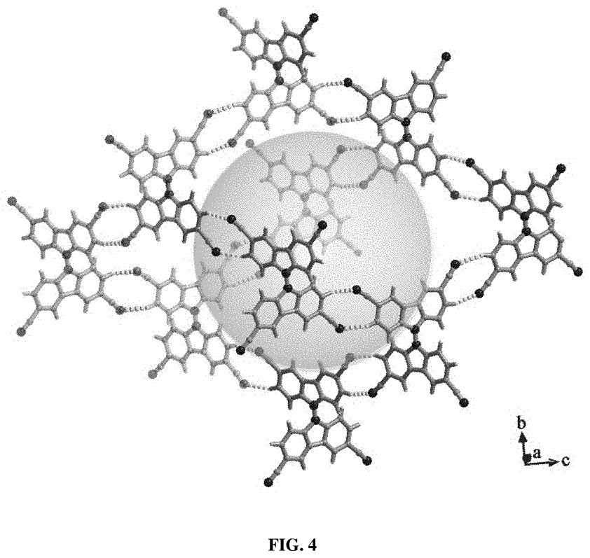

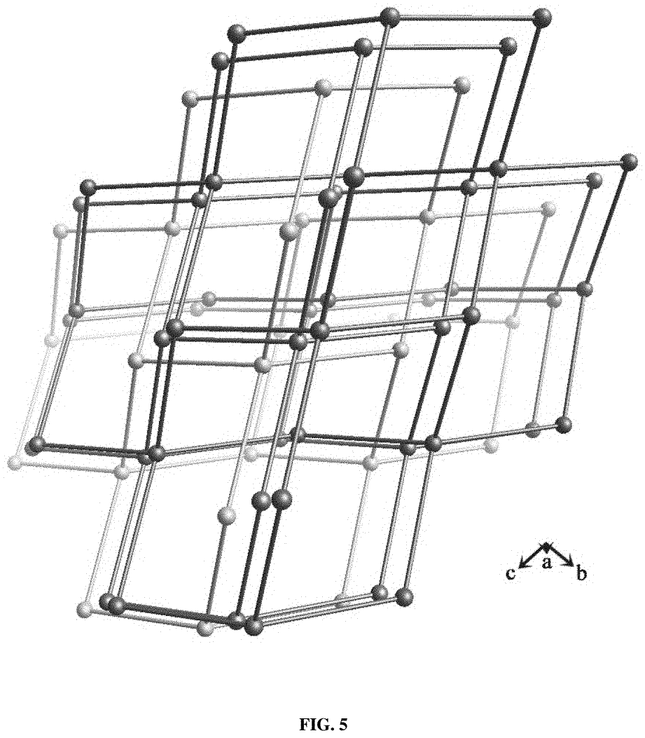

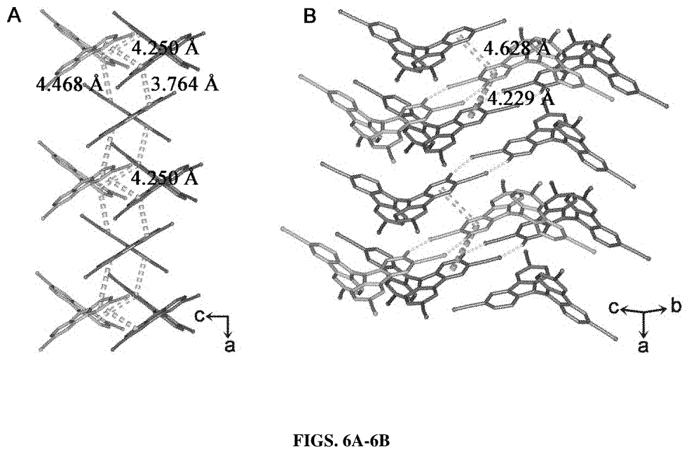

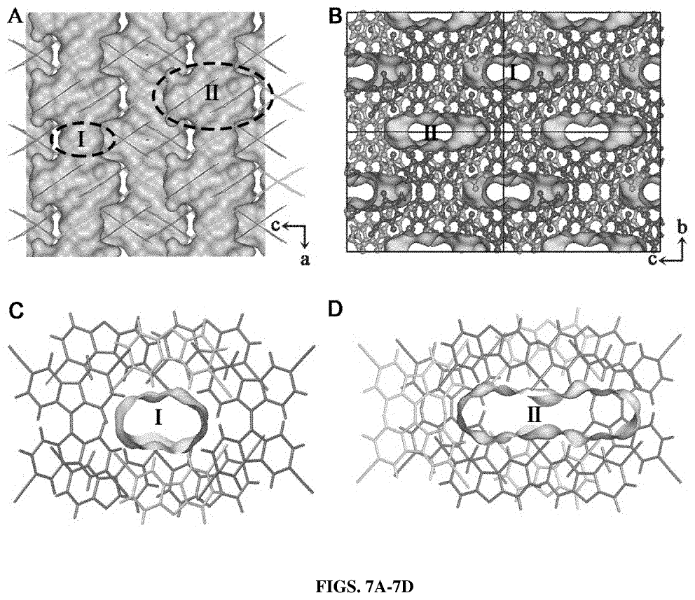

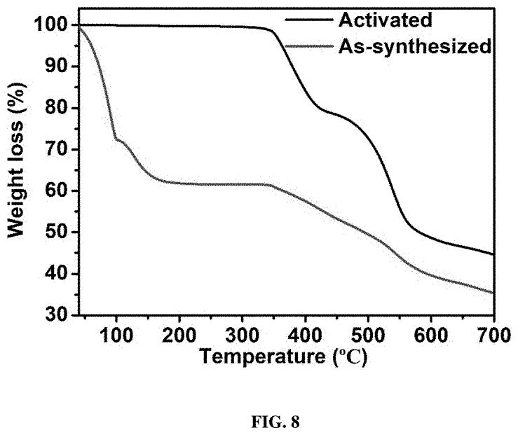

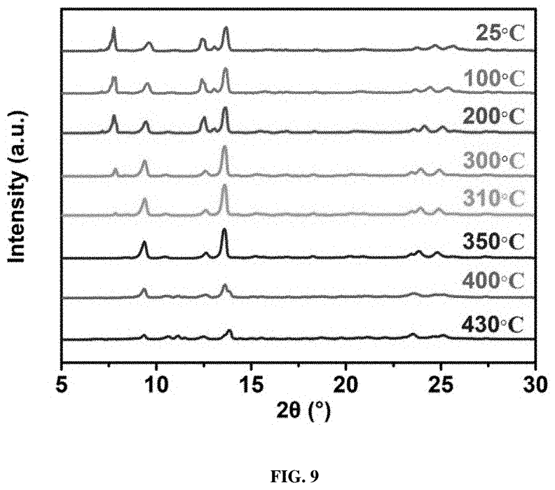

The organic ligand was obtained from the cyanolization of 3,3′6,6′-tetrabromo-9,9′-bicarbazole after the oxidative coupling reaction of 3,6-dibromocarbazole (FIG. 2A), which was confirmed by IR, ¹H NMR, ¹³C NMR spectroscopy, elemental analysis and thermogravimetric analysis. The high-quality single crystals were then achieved from the hot saturated N,N′-dimethylformamide (DMF) solution of HOF-FJU-1 powders via recrystallization. Single-crystal X-ray diffraction studies indicated that HOF-FJU-1 crystallized in orthorhombic space group Pnn2 (FIG. 3 and Table 1). In this HOF, there were three crystallographically different bi-carbazole molecules in its asymmetric units. Each bi-carbazole unit was linked to four neighbor bi-carbazoles by four pairs of CN . . . H—C hydrogen bonds to form a single dia network (FIG. 2B, 2C and FIG. 4). The space of the single network allows to form a three-fold interpenetrating array, showing distinct offset π . . . π interactions along a axis with distances of 3.764-4.628 Å that may further enhance the rigidity of this type of flexible porous materials (FIG. 5 and FIG. 6A-6B). After multiple interpenetration, there were still 1D channels running along the crystallographic [100] direction, with alternating large cages and small necks, showing void pore volume of about 20% of the total crystal cell. Taking the van der Waals radius into account, the aperture size in the pore channel was ca. 3.41×5.29 Å2 (FIG. 2D and FIG. 7), corresponding to molecular size of ethylene. The thermal stability of HOF-FJU-1 was investigated via thermogravimetric analysis (TGA) and variable-temperature powder X-ray diffraction (PXRD) in N₂ atmosphere (FIG. 8 and FIG. 9), indicating that this HOF can retain its framework upon exposure at >300° C. Notably, after vacuuming the as-synthesized sample at 150° C. for 12 h, the crystal structure of guest-free HOF-FJU-1a was directly obtained from X-ray diffraction analysis through a single-crystal to single-crystal transformation manner, indicating the robustness of HOF-FJU-1 during desolvation. The weak intermolecular interactions, framework and pore structures were well conserved after guest removal, with only slight changes (increase of <7%) in parameters of crystal unit-cell (Table 1). Accordingly, the robust pore space with appropriate size rendered this HOF a potential adsorbent for ethylene/ethane separation.

TABLE 1
Crystallographic Data and Structural Refinement Summary.

| Compounds | HOF-FJU-1 | HOF- FJU⊃H₂0^{(b)} | HOF-FJU⊃C₂H₄ |
|---|---|---|---|
| CCDC | 1878390 | 1871845 | 1942488 |
| Empirical formula | C₄₂H₁₈N₉ | C₄₂H₁₈N₉O | C_{42.75}H_{19.5}N₉ |
| Formula weight | 648.65 | 664.65 | 665.66 |
| Temperature (K) | 293 | 293 | 100 |
| Crystal system | orthorhombic | orthorhombic | orthorhombic |
| Space group | Pnn2 | Pnn2 | Pnn2 |
| a (Å) | 12.5365(9) | 12.4635(3) | 12.0688(5) |
| b (Å) | 15.1402(11) | 14.2649(4) | 14.4808(13) |
| c (Å) | 19.7683(15) | 19.7556(5) | 19.9249(8) |
| Volume (Å³) | 3752.1(5) | 3512.37(15) | 3482.2(4) |
| Z | 4 | 4 | 4 |
| D_{c} (g cm⁻³) | 1.148 | 1.257 | 1.27 |
| μ (mm⁻¹) | 0.570 | 0.080 | 0.079 |
| F(000) | 1332.0 | 1364.0 | 1371.0 |
| Crystal size (mm³) | 0.03 × 0.05 × 0.18 | 0.04 × 0.05 × 0.2 | 0.03 × 0.05 × 0.18 |
| Radiation | Cu Kα | Cu Kα | Cu Kα |
| | (λ = 1.54178 Å) | (λ = 1.54178 Å) | (λ = 1.54178 Å) |
| Goodness-of-fit on F² | 1.068 | 1.054 | 1.039 |
| Final R indexes [I >= 2σ (I)](^{a)} | R₁ = 0.0983, | R₁ = 0.0697, | R₁ = 0.0682, |
| | wR₂ = 0.2880 | wR₂ = 0.2046 | wR₂ = 0.1619 |
| Final R indexes [all data]^{(a)} | R₁ = 0.1244, | R₁ = 0.0828, | R₁ = 0.1149, |
| | wR₂ = 0.3262 | wR₂ = 0.2301 | wR₂ = 0.1882 |

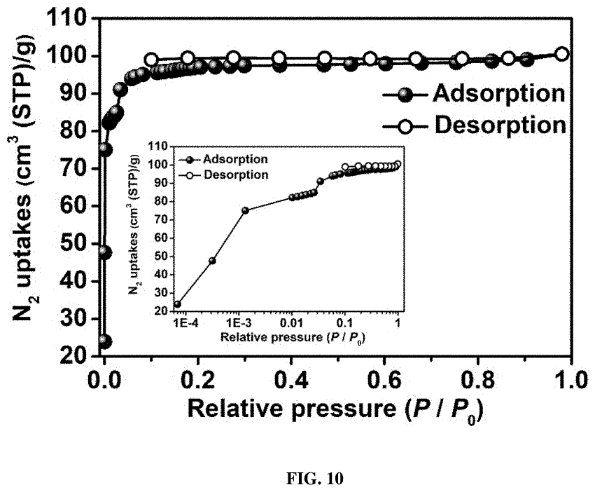

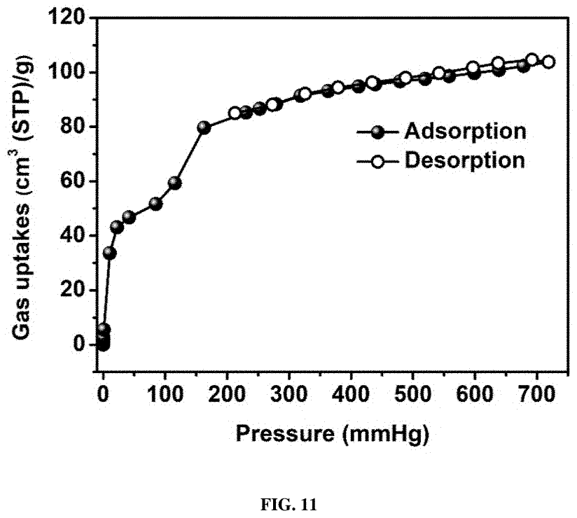

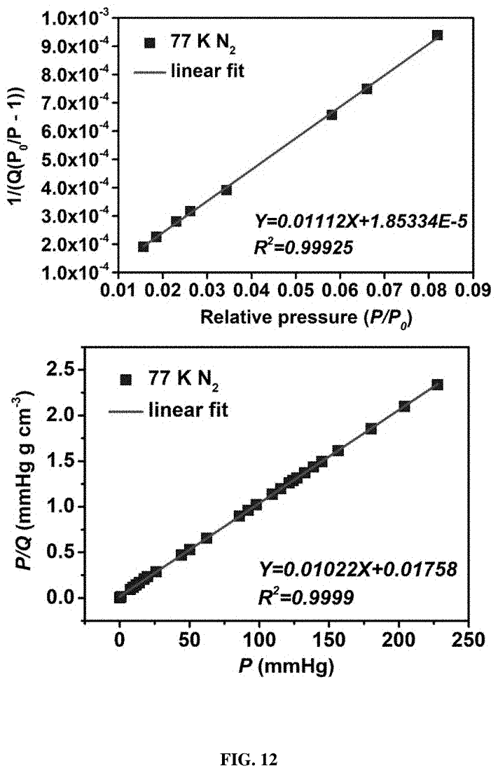

To verify the porosity of HOF-FJU-1a, the N₂ sorption at 77 K was carried out (FIG. 10). The stepwise adsorption isotherm indicated flexible-robust feature of HOF-FJU-1a, showing steep N₂ adsorption into the initial robust pore channels until saturation that was followed by a further uptake because of the slightly flexible host. The adsorption amount of N₂ at the first step was 83 cm³ g⁻¹ at P/P₀=0.001, corresponding to a pore volume of −0.13 cm³ g⁻¹. The total N₂ uptake at 77 K and 1 bar was 100 cm³ g⁻¹, corresponding to a total pore volume of −0.15 cm³ g⁻¹, which is in line with the theoretical pore volume calculated from the crystal structure (0.17 cm³ g⁻¹). Similar stepwise adsorption isotherm was also observed for CO₂ in HOF-FJU-1a at 195 K and 1 bar (FIG. 11). The experimental Brunauer-Emmett-Teller and Langmuir surface area calculated from N₂ adsorption isotherms were 390 and 426 m² g⁻¹, respectively (FIG. 12).

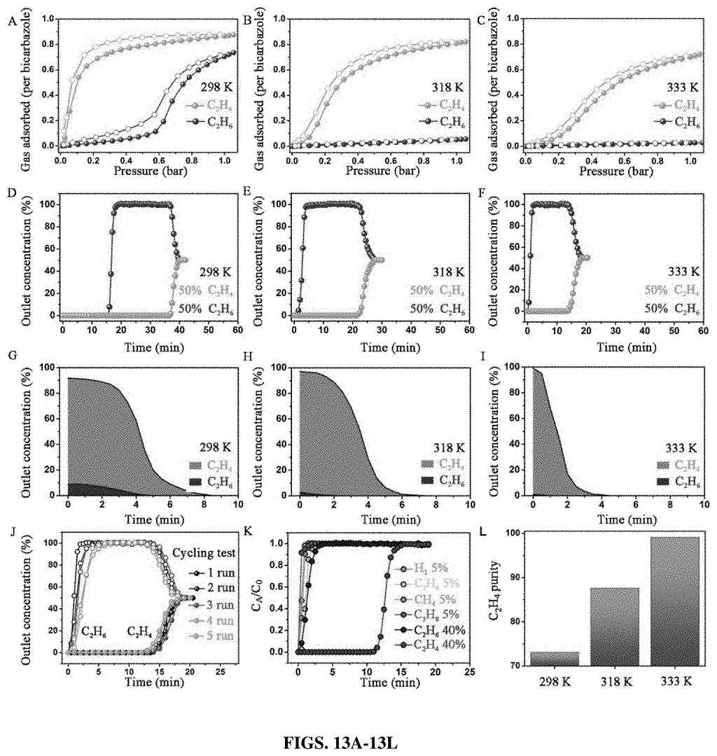

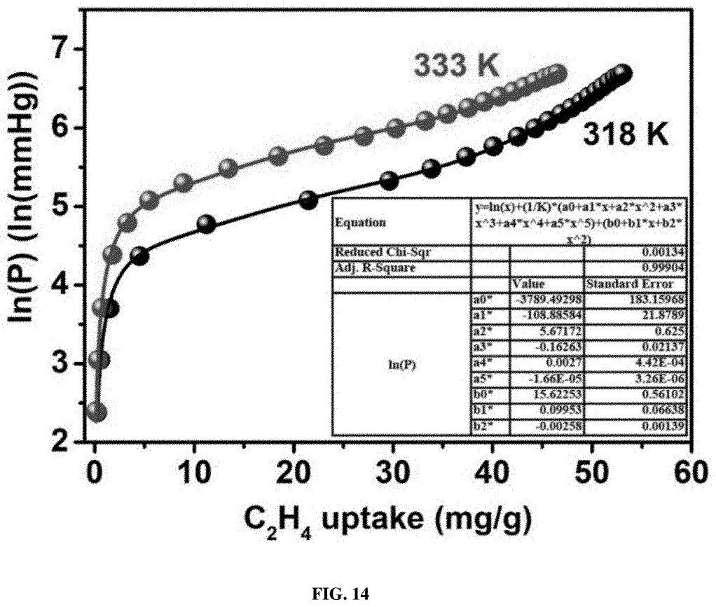

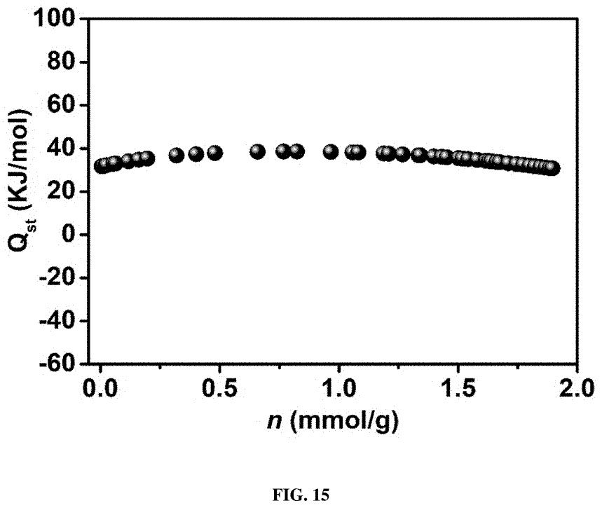

The unique flexible-robust feature encountered in HOF-FJU-1a was evaluated with respect to its effect on C₂H₄/C₂H₆ separation. Single-component static adsorption isotherms for C₂H₄ and C₂H₆ were collected at ambient conditions. At 298 K, the C₂H₄ sorption isotherm of HOF-FJU-1a showed a distinct sharp step at relatively low pressures (FIG. 13A), indicating the well accommodation of the compact pore space in this HOF for C₂H₄ molecules, which promoted the adsorption equilibrium even at low pressures and gas concentrations. The total C₂H₄ uptake at 1 bar and 298 K was 47.4 cm³ g⁻¹ (2.12 mmol g⁻¹). Surprisingly, HOF-FJU-1a showed a negligible C₂H₆ uptake when the dosing pressure was below ˜0.58 bar, followed by a steep rise after gate-opening with a total C₂H₆ uptake of 38.5 cm³ g⁻¹ at 1 bar. These results indicated that HOF-FJU-1a showed high sieving separation for C₂H₄/C₂H₆ at relatively low pressures. Given that the gate pressures can be promoted at elevated temperatures, the sorption behavior of HOF-FJU-1a for C₂H₄ and C₂H₆ at 318 and 333 K (FIGS. 13B and 13C) was investigated. Indeed, owing to the increased gate-opening pressures, at 333 K and 1 bar, HOF-FJU-1a showed a negligible uptake of 1.4 cm³ g⁻¹ (0.06 mmol g⁻¹) for C₂H₆, lower than that of UTSA-280 (0.10 mmol g⁻¹) upon sieving separation (Lin et al., 2018), whereas steadily C₂H₄ adsorption can be still observed with a total uptake of 36.2 cm³ g⁻¹ (1.62 mmol g⁻¹). The loading dependent isosteric heats of C₂H₄ in HOF-FJU-1a was derived from the adsorption isotherms at different temperatures, giving a moderate value of 31.6 kJ mol-1 (FIG. 14 and FIG. 15). Notably, these results revealed the great potential of high sieving separation for C₂H₄/C₂H₆ by directly adjusting the adsorption gate-pressures in the flexible-robust HOF-FJU-1a.

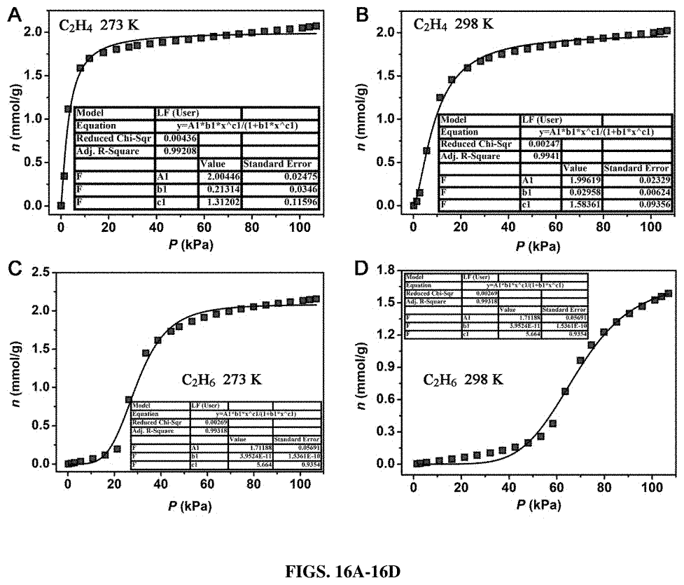

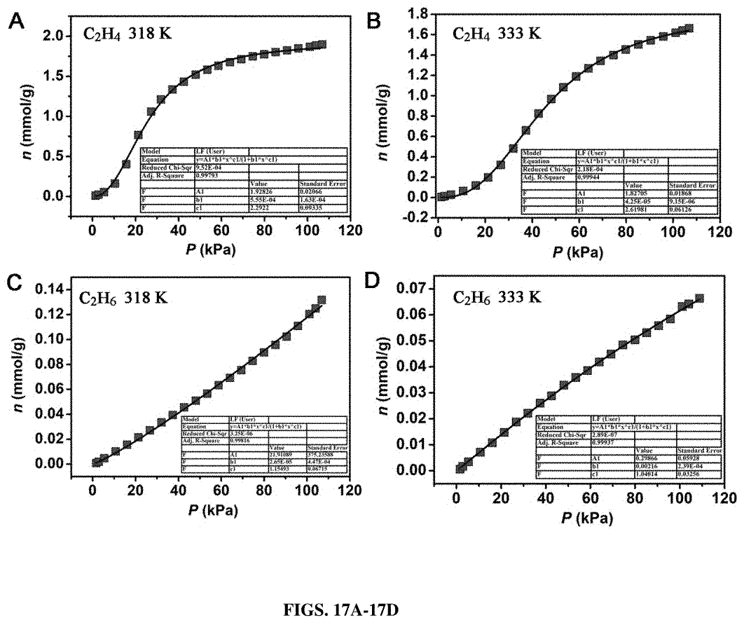

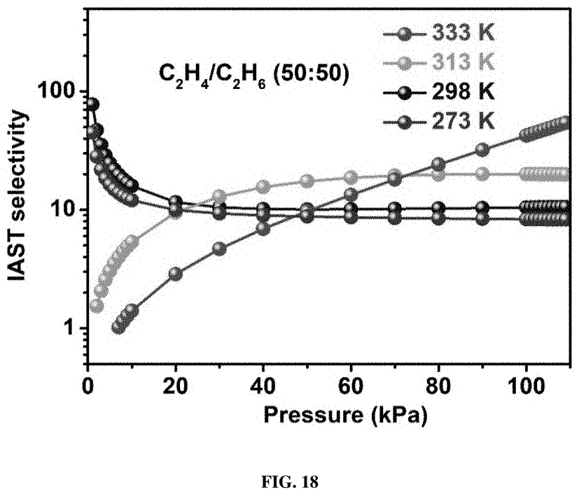

Ideal adsorbed solution theory (IAST) calculations were then employed to estimate the C₂H₄/C₂H₆ (50:50, v:v) separation selectivity at different temperatures. At 298 K, HOF-FJU-1a showed a moderate C₂H₄/C₂H₆ selectivity of 10.5 because of the co-adsorption of C₂H₆. Upon the increasing of temperatures (the co-adsorption of C₂H₆ is greatly suppressed), the C₂H₄/C₂H₆ selectivity of HOF-FJU-1a was promoted to 42.3 at 333 K and 1 bar (FIG. 16 to FIG. 18), which was significantly larger than those of the benchmark Fe-MOF-74 (13.6) (Bloch et al., 2012) and Zeolite 5A (4.5) (Mofarahi et al., 2013). It should be noted that, for those porous materials of high sieving effect, the apparent C₂H₆ adsorption of ultra-low uptake is often subject to large measurement error that leads to significant uncertainties in the estimated selectivity. Therefore, the IAST selectivity is valid only for the qualitative comparison. Although the high C₂H₄/C₂H₆ selectivity of HOF-FJU-1a is sufficient for high purity ethylene capture, corresponding breakthrough tests in a fixed-bed column are prerequisite to validate the production purity of C₂H₄.

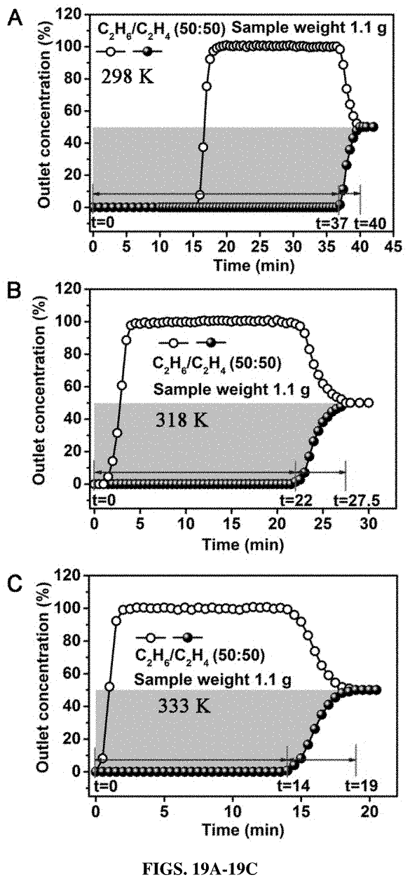

Ethylene is one of the most important feedstocks in the chemical industry for the production of rubbers, plastics, fuel components and other valuable chemical products, showing a global annual ethylene demand of over 160 million tons. In the common process for ethylene production based on the cracking of heavier hydrocarbon fractions, followed by dehydrogenation reactions, the conversion yield of the later step is only around 50-60%. Other processes such as catalytic dehydrogenation also give an equimolar mixture of C₂H₄ and C₂H₆. Thus, the upgrading of ethylene from the C₂H₄/C₂H₆ mixture is an important process before further utilization. Traditionally, separating ethylene from ethane requires cryogenic distillation, an energy-intensive step. To confirm the actual C₂H₄/C₂H₆ separation performance of HOF-FJU-1a, fixed-bed breakthrough tests were conducted at 298-333 K, in which the mixture of C₂H₄/C₂H₆ (50:50, v/v) was flowed over a packed column of activated HOF-FJU-1a sample with a total flow of 1.25 mL/min at different temperatures (FIG. 13D to FIG. 13F). At 298 K, despite achievement of remarkable C₂H₄ capture, there was a distinct C₂H₆ co-adsorption in the packed column of HOF-FJU-1a as indicated by a moderate retention time for C₂H₆ in the breakthrough curves. This is because the partial pressure of C₂H₆ in the gas mixture reached the borderline of gate-opening. Therefore, the production purity of C₂H₄ at 298 K was only 73.1% as calculated from the concentration curves in the regeneration step (FIG. 13G). In contrast, by tuning the gate-pressures to minimize co-adsorption of C₂H₆, the neat breakthrough curves of HOF-FJU-1a at 333 K were then achieved, which showed an immediate C₂H₆ elution without any detectable C₂H₄, and a remarkable C₂H₄ retention prior to the breakthrough of C₂H₄. Thus, the C₂H₄ purity was significantly boosted up to 99.1% at 333 K (FIG. 13H and FIG. 13I), which was higher than those performed by ITQ-55 (Bereciartua et al., 2017) and Cu(OPTz) (Gu et al., 2019), and almost identical to that of UTSA-280 (99.2%) (Lin et al., 2018). The captured C₂H₄ amount from the equimolar C₂H₄/C₂H₆ breakthrough curve at 333 K was calculated to be 0.407 mol kg-1 (FIG. 19). These results demonstrated that HOF-FJU-1a is competent to realize the high C₂H₄/C₂H₆ sieving separation under mild conditions. To evaluate the durability, multiple cycling breakthrough experiments under same conditions were performed, showing that HOF-FJU-1a retained its captured C₂H₄ productivity as the first cycle (FIG. 13J). The effect of potential gas impurity in realistic conditions was also investigated as exemplified by breakthrough study on a gas stream of H₂/C₃H₆/CH₄/C₃H₈/C₂H₆/C₂H₄ (5:5:5:5:40:40 v/v/v/v/v/v). In this context, HOF-FJU-1a still exclusively captured C₂H₄ from the mixture (FIG. 13K). Overall, these results demonstrated that high sieving separation of C₂H₄ from relevant mixtures were realized in HOF-FJU-1a. It should be noted that the current industrial separation purification of ethylene is achieved by energy-intensive process involving repeated distillation-compression cycling under harsh conditions in a huge splitter column, which consumes up to about 800 PJ, more than 0.3% of annual global energy consumption (Sholl and Lively, 2016). To fully capture C₂H₄ in the hot gas stream from cracking tower (typically up to 50° C.), HOF-FJU-1a could serve as promising adsorbent for energy-efficient separation as demonstrated here.

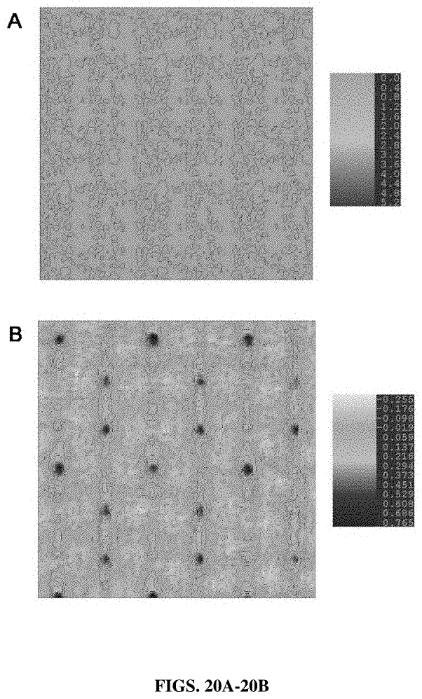

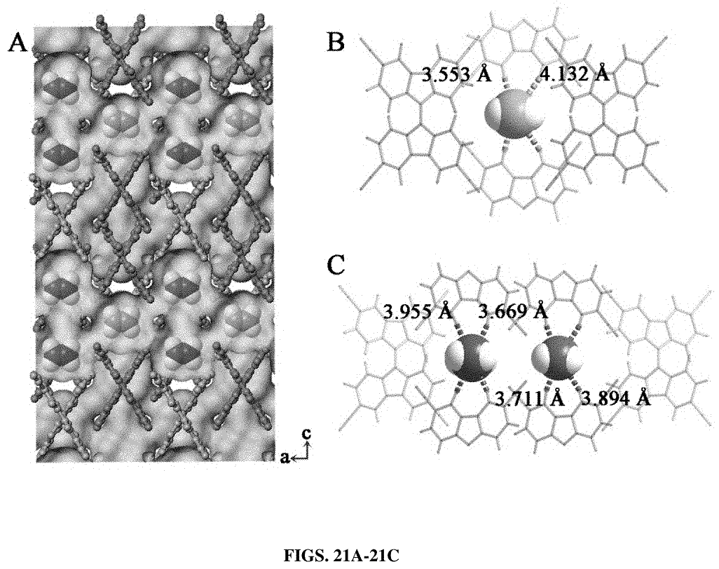

To structurally understand the interactions of C₂H₄ molecules within HOF-FJU-1a, single-crystal X-ray diffraction measurements of C₂H₄-loaded sample were carried out to determine the binding conformations of C₂H₄. The data for HOF-FJU-1.0.75 C₂H₄ were collected at 100 K, from which the location of C₂H₄ molecules were successfully identified as indicated by a remarkable increase on residual electron intensity (FIG. 20 and Table 1). As shown in FIG. 21A, there were three crystallographically different C₂H₄ molecules in the compact pore channel. The C₂H₄ molecules were well dispersed along the 1D pore channel with only host-guest interactions observed. Multiple intermolecular interactions, mainly C-H·π with the framework (3.553-4.132 Å), were shown. Notably, compared with the guest-free HOF-FJU-1a, the C₂H₄-loaded structure showed a structural shrinkage of 7.2% in the unit cell volume (Table 1). Without wishing to be bound by theory, this effect is likely due to the formation of host-guest interactions that hold the framework tightly together. Such slight structural transformation is desirable during C₂H₄ capture from gas mixture, as the structural shrinking can downsize the pore structure for inward diffusion of C₂H₆, thus further enhancing the sieving effect.

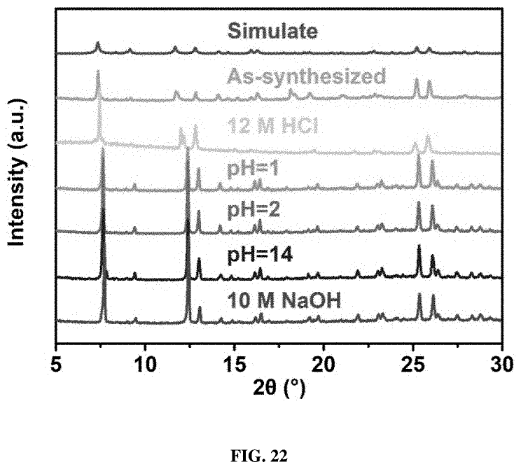

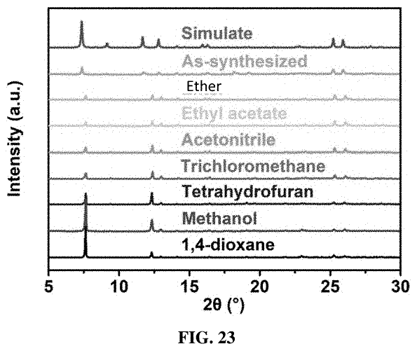

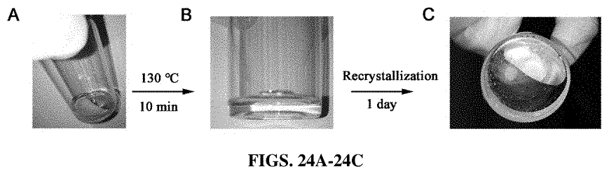

Apart from excellent C₂H₄/C₂H₆ separation performance, HOF-FJU-1 exhibited excellent chemical stability as validated by PXRD of samples upon exposure to different harsh conditions. In these tests, HOF-FJU-1 retained its crystallinity in aqueous solutions with the pH value ranging from 1 to 14, even in solutions like 12 M HCl or 10 M NaOH (FIG. 22). Similar phenomena were observed for HOF-FJU-1 immersed in various organic solvents including CH₂C₁₂, hexanes, toluene, and acetone, except in N,N′-diethylformamide and N,N′-dibutylformamide (FIG. 23). Furthermore, the HOF-FJU-1 can be quickly regenerated from the N,N′-Dimethylformamide (DMF) (FIG. 24). It should be noted that almost all reported stable HOFs are labile to basic solution owing to their deprotonation or hydrolysis. Here, without being bound by theory, the strong intermolecular interactions and compact structure jointly endowed HOF-FJU-1 with excellent thermal and chemical stability in contrast to those HOFs with groups such as amides, carboxylic acids (Table 2). Compared with MOFs, HOFs are also much more easily processed into different types of forms such as spheres and membranes for the large-scale fixed bed and membrane gas separations, respectively.

TABLE 2
Summary of chemical and thermal stability of different functional groups

| | Chemical | Thermal | |
|---|---|---|---|
| Functional group | Stability | Stability | Ref. |
| 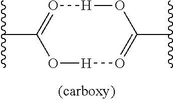 | 12M HCl | 300- 400° C. | Yin et al., 2018; Hu et al., 2017 |
| 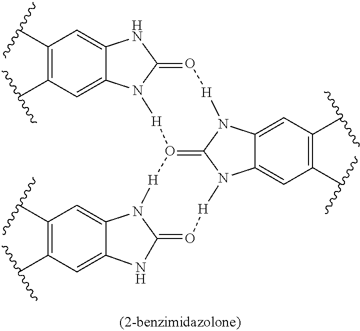 | — | 350- 440° C. | Mastalerz and Oppel, 2012; Yan et al., 2017 |
| 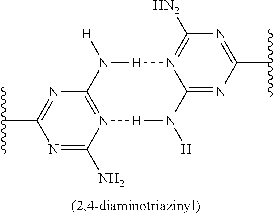 | — | 350- 420° C. | Li et al., 2015; Wang et al., 2015; Li et al., 2014 |
| 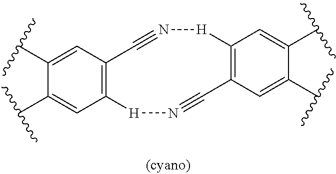 | 12M HCl- 10M NaOH | 300° C. | Described herein |

### Example 2: Synthesis and Characterization of HOF-FJU-1

3,6-dibromocarbazole (99%, HWG), potassium permanganate (99.5%, SCRC), acetone (99.5%, SCRC), Cuprous cyanide (99.5%, SCRC), Anhydrous ferric chloride (98%, Aldrich), anhydrous dimethylformamide (DMF, 99%, Sigma-Aldrich), were purchased and used without further purification.

N₂ (99.999%), C₂H₄ (99.99%), C₂H₆ (99.99%), He (99.999%), C₂H₆/C₂H_{4=50/50} (v/v), H₂/C₃H₆/CH₄/C₃H₈/C₂H₆/C₂H₄/(5/5/5/5/40/40 v/v/v/v/v) were purchased from Beijing Special Gas Co. LTD (China).

3,3′6,6′-tetrabromo-9,9′-bicarbazole: To a solution of 3,6-dibromocarbazole (1.625 g, 5 mmol) in 25 mL acetone, potassium permanganate (2.37 g, 15 mmol) was added at 50° C. and then the solution was stirred for 5 h at 60° C. with a reflux condenser and cooled down to room temperature. After removal of the organic solvents, the residue was extracted with CHCl₃ (250 mL) for 12 h with stirring. The filtrate was washed three time with CHCl₃. The residue was purified by recrystallization from chloroform/hexane to give a colorless crystal. (0.83 g 51% yield). ¹H NMR (400 MHz, DMSO-d6): δ=8.72 (d, 4H, J=1.6 Hz), 7.5 (dd, 4H, J=2, 1.6 Hz), 6.9 (d, 4H, J=8.8 Hz) ppm.

3,3′6,6′-tetracyano-9,9′-bicarbazole: A mixture of CuCN (2.782 g, 31.06 mmol) and compound 3,3′6,6′-tetrabromo-9,9′-bicarbazole (2 g, 2.1 mmol) in dry DMF (50 mL) was added to a 120 mL Schleck flask charge with stir bar at 150° C. for 48 h under nitrogen atmosphere. After cooling to room temperature, the reaction mixture was treated with concentrated HCl (40 mL) and Iron (III) trichloride (30 g, 184.9 mmol) the solution stirred at 0° C. for 2 h. The reaction mixture was diluted with water (200 mL), filtered and the gray colored solid collected (1.3 g, 97% yield). ¹H NMR (400 MHz, DMSO-d6): δ 8.95 (s, 4H), 7.67 (d, 4H, J=8.4 Hz), 7.55 (d, 4H, J=8.8 Hz) ppm; ¹³C NMR (400 MHz, DMSO-d6) δ=142.33, 132.57, 128.00, 122.36, 120.01, 111.48, 106.00 ppm; FT-IR (cm⁻¹) 2225 (vc_{N}), 1602, 1485, 1451, 1365, 1292, 1238, 1186, 1137, 1028, 893, 815, 587. The compound was best formulated as HOF-FJU-1·DMF·10H₂O TGA data: Calcd. weight loss for 10H₂O and one DMF molecules: 33.44%, Found: 35.60%; Anal. Calcd. for C₄₈H₅₂N₁₁₀₁₂: C, 59.07; N, 15.79; found: C, 59.31; N, 15.82%.

Crystallization of the organic building block (HOF-FJU-1). The organic building block (0.1 g, 0.23 mmol) was dissolved in DMF (2 mL) under 130° C. with glass flask. The resulting solution was cooled to room temperature. The bottle was then kept at room temperature (23° C.) for one night. Colorless needle-like crystals were obtained. The PXRD results showed that, the organic building block (HOF-FJU-1) is a pure phase (FIG. 3).

Sample characterization. The crystallinity and phase purity of the samples were measured using powder X-ray diffraction (PXRD) with a Rigaku Ultima IV X-ray Diffractometer with Cu-Kα radiation (λ=1.54184 Å), with a nitrogen atmosphere, scanning over the range 5-30°. The Fourier transform infrared (KBr pellets) spectra was recorded in the range of 400-4000 cm⁻¹ on Thermo Nicolet 5700 FT-IR instruments. ¹H NMR and ¹³C NMR experiments were performed on Bruker Advance III 400 MHZ. Thermogravimetric analyses (TGA) were performed with METTLER Q50 under nitrogen atmosphere with a heating rate 10° C. min⁻¹ from 40 to 700° C., A Micromeritics ASAP 2020 surface area analyzer was used to measure gas adsorption isotherms. To have a guest-free framework, the fresh sample was filtered out and vacuumed at room temperature for 24 h followed by 150° C. until the outgas rate was 5 mmHg min⁻¹ prior to measurements. A sample was used for the sorption measurement and maintained at 77 K with liquid nitrogen, at 273, 298, 318 and 333 K ethane and ethylene. The single-crystal X-ray was performed with Agilent Technologies SuperNova A diffractometer and the structure were solved by direct methods and refined by full matrix least-squares methods with the SHELX program package. MALDI mass spectra were obtained from Bruker MALDI-TOF mass spectrometer. Elemental analyses were performed on a Vario EL III analyzer.

### Example 3: Adsorption Enthalpies, IAST Calculations, and Breakthrough Experiment

The isosteric enthalpies of adsorption (Qₛₜ). Using the data collected of C₂H₄ and C₂H₆ at 318 K and 333 K, the isosteric enthalpy of adsorption was calculated. The data was fitted using a virial-type expression composed of parameters aᵢ and bᵢ (eq. 1). Then, the Qₛₜ (kJ mol⁻¹) was calculated from the fitting parameters using (eq. 2), where p is the pressure (mmHg), T is the temperature (K), R is the universal gas constant (8.314 J·mol⁻¹·K⁻¹), Nis the amount adsorbed (mg g⁻¹), and m and n determine the number of terms required to adequately describe the isotherm.

The virial equation be written as follows:

$$ln p = ln N + \frac{1}{T} {\sum }_{i = 0}^{m} {a}_{i} {N}_{i} + {\sum }_{i = 0}^{n} {b}_{i} {N}_{i} \qquad ( 1 )$$

The calculation formula for isosteric enthalpies of adsorption:

$${Q}_{st} = - R {\sum }_{i = 0}^{m} {a}_{i} {N}_{i} \qquad ( 2 )$$

Prediction of the Gas Adsorption Selectivity by IAST. Fitting details: the adsorption data for C₂H₄ and C₂H₆ in HOF-FJU-1 at 273, 298, 318 and 333 K were fitted with single-site Langmuir-Freundlich equation.

$$N = {N}^{max} \times N \frac{{bp}^{1 / n}}{1 + {bp}^{1 / n}} \qquad ( 3 )$$

where p is the pressure of the bulk gas in equilibrium with the adsorbed phase (kPa), N is the amount adsorbed per mass of adsorbent (mmol g⁻¹), N^{max} is the saturation capacities of site 1 (mmolg⁻¹), b is the affinity coefficients of site 1 (1/kPa) and n represents the deviations from an ideal homogeneous surface.

IAST calculation: The adsorption selectivity based on IAST for C₂H₄/C₂H₆ mixed is defined by the following equation:

$${S}_{A / B} = \frac{{q}_{A} {y}_{B}}{{q}_{B} {y}_{A}} \qquad ( 4 )$$

where xᵢ and yᵢ are the mole fractions of component i (i=A, B) in the adsorbed and bulk phases, respectively.

Breakthrough Experiment. The breakthrough experiments for C₂H₄/C₂H₆ (50:50, v/v) gas mixtures were carried out at a flow rate of 1.25 mL/min. Activated HOF-FJU-1 powder (1.1 g) was packed into ϕ3×300 mm stainless steel column. The experimental set-up consisted of two fixed-bed stainless steel reactors. One column was loaded with the adsorbent and kept at different temperature (298, 318 and 333 K), while the other reactor was used as a blank control group to stabilize the gas flow. The flow rates of all gases mixtures were regulated by mass flow controllers, and the effluent gas stream from the column is monitored by a gas chromatography (TCD-Thermal Conductivity Detector, detection limit 0.1%). Prior to the breakthrough experiment, we flushed the activated sample in adsorption bed with helium gas (100 mL/min) for 30 min at 333 K to ensure the totally removal of adsorbed gas.

All of the compounds, material, compositions, and methods disclosed and claimed herein can be made and executed without undue experimentation in light of the present disclosure. While the disclosure may have focused on several embodiments or may have been described in terms of preferred embodiments, it will be apparent to those of skill in the art that variations and modifications may be applied to the compounds, compositions, and methods without departing from the spirit, scope, and concept of the invention. All variations and modifications apparent to those skilled in the art are deemed to be within the spirit, scope, and concept of the invention as defined by the appended claims.

### REFERENCES

The following references, to the extent that they provide exemplary procedural or other details supplementary to those set forth herein, are specifically incorporated herein by reference.
WO 2019/183635
Aguado et al., J. Am. Chem. Soc., 134:14635-14637, 2012.
Anderson, Practical Process Research & Development—A Guide for Organic Chemists (2012).
Anson et al., Chem. Eng. Sci., 63:4171-4175, 2008.
Bachman et al. J. Am. Chem. Soc., 139:15363-15370, 2017.
Bao et al., Energy Environ. Sci., 9:3612-3641, 2016.
Bao et al., Angew. Chem. Int. Ed., 130(49):16252-57, 2018.
Barone et al., J. Comput. Chem. 30, 934-939, 2009.
Bereciartua et al., Science, 358:1068-1071, 2017.
Bloch et al., Science, 335:1606-1610, 2012.
Cadiau et al., Science, 353:137-140, 2016.
Chang et al., Chem. Commun. 51:2859-2862, 2015.
Chu et al., Nat. Mater., 16:16-22, 2017.
Crawford et al., Chem. Sci., 6(3):1645-49, 2015.
Cui et al., Science, 353:141-144, 2016.
Cui et al., Inorganica Chimica Acta, 495:118938, 2019.
Faiz et al., Chem. Eng. Sci., 73:261-284, 2012.
Friščič et al., Angew. Chem. Int. Ed., 49(4):712-15, 2010.
Furukawa et al., Science 341(6149):1230444, 2013.
Geier et al., Chem. Sci. 4:2054-2061, 2013.
Giannozzi et al., J. Phys.: Condens. Matter 21:395502, 2009.
He et al., Energy Environ. Sci. 5:9107-9120, 2012.
Horike et al., J. Am. Chem. Soc., 135:4612-4615, 2013.
Hu et al., Angew. Chem. Int. Ed., 56:2101-2104, 2017.
Ji et al., J. Am. Chem. Soc., 139:11325-11328, 2017.
Julien et al., J. Am. Chem. Soc., 138(9):2929-32, 2016.
Kishida et al., Angew. Chem. Int. Ed. 55:13784-13788, 2016).
Kitagawa, Angew. Chem. Int. Ed., 54:10686-10687, 2015.
Klet et al., J. Am. Chem. Soc. 137:15680-15683, 2015.
Krishna et al. Microporous Mesoporous Mater. 185:30-50, 2014.
Li et al., Chem. Soc. Rev., 38(5):1477-1504, 2009.
Li et al., J. Am. Chem. Soc., 136:547-549, 2014.
Li et al., J. Am. Chem. Soc., 136:8654-8660, 2014.
Li et al., Chemistry—A European Journal, 21(18):6913-20, 2015.
Li et al., Angew. Chem. Int. Ed., 54:574-577, 2015.
Li et al., Science, 362(6413):443-46, 2018.
Li et al., Angew. Chem. Int. Ed., 130(46):15403-08, 2018.
Liao et al., Science, 356:1193-1196, 2017.
Lin et al., Chem. Commun., 47(32):9185-87, 2011.
Lin, Science, 353:121-122, 2016.
Lin et al., J. Am. Chem. Soc., 139(23):8022-28, 2017.
Lin et al., Nat. Mater., 17(12):1128, 2018.
Lin et al., J. Am. Chem. Soc., 140(40):12940-46, 2018.
Lin et al., Coord. Chem. Rev., 378:87-103, 2019.
Lin et al., Coord. Chem. Rev., 384:21-36, 2019.
Lin et al., Chem, 6(2):337-63, 2020.
Liu et al., Chem. Soc. Rev., 47(12):4642-64, 2018.
Ma et al., J. Am. Chem. Soc., 131:6445-6451, 2009.
Ma et al., Science, 361(6406):1008-11, 2018.
Majano et al., Adv. Mater., 25(7):1052-57, 2013.
Mastalerz et al., Angew. Chem. Int. Ed., 51:5252-5255, 2012.
Maserati et al., Chem. Mater., 28(5):1581-88, 2016.
Mofarahi et al., Adsorption, 19:101-110, 2013.
Mottillo et al., Green Chemistry, 19(12):2729-47, 2017.
Nugent et al., Nature, 495:80-84, 2013.
Pan et al., Angew. Chem. Int. Ed., 45:616-619, 2006.
Peng et al., Science, 346:1356-1359, 2014.
Robl et al., Mater. Res. Bull., 22:373-380, 1987.
Rubio-Martinez et al., Chem. Soc. Rev., 46(11):3453-80, 2017.
Sen et al., J. Am. Chem. Soc., 139:18313-18321, 2017.
Shi et al., CrystEngComm, 19(30):4265-68, 2017.
Sholl et al., Nature, 532:435-437, 2016.
Stojaković et al., Chem. Commun., 48(64):7958-60, 2012.
Vaidhyanathan et al., Science, 330:650-653, 2010.
Wang et al., J. Am. Chem. Soc., 137:9963-9970, 2015.
Webster et al., J. Am. Chem. Soc., 120:5509-5516, 1998.
Yan et al., Chem. Commun., 53:3677-3680, 2017.
Yang et al., Nat. Chem. 7:121-129, 2014.
Yang, in Adsorbents: Fundamentals and Applications. (John Wiley & Sons, Inc.), pp. 280-381, 2003.
Yin et al., Angew. Chem. Int. Ed., 130:7817-7822, 2018.
Yoon et al., Angew. Chem. Int. Ed., 49:5949-5952, 2010.
Yoon et al., Nat. Mater., 16:526-531, 2016.
Yuan et al., Angew. Chem. Int. Ed., 49(23):3916-19, 2010.
Yuan et al., CrystEngComm, 12(12):4063-65, 2010.
Zhai et al., Nat. Commun., 7:13645, 2016.
Zhao et al., J. Am. Chem. Soc., 137(43):13756-59, 2015.
Zhang et al., Chem. Commun. 51:2714-2717, 2015.
Zhang, et al., Energy & Fuels, 22:1142-1147, 2008.
Zheng et al., Inorg. Chem., 57:9096, 2018.

What is claimed is:

1. A compound of the formula: 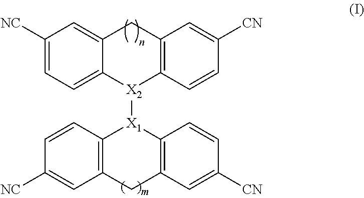
wherein:
X₁ and X₂ are each independently CH or N; and
m and n are each independently 0, 1, 2, or 3.

2. The compound of claim 1 further defined as:

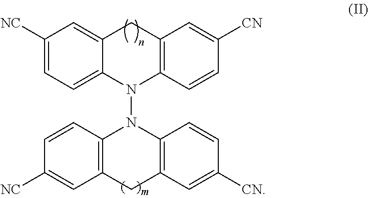

3. The compound of claim 1 further defined as:

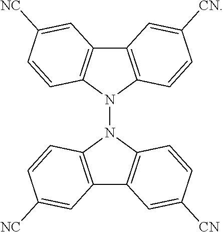 [SMILES: N#Cc1ccc2c(c1)c1cc(C#N)ccc1n2-n1c2ccc(C#N)cc2c2cc(C#N)ccc21]

4. A framework comprising a repeating unit of a compound of claim 1.

5. The framework of claim 4, wherein the repeating units are joined by non-covalent interactions.

6. The framework of claim 4, wherein the non-covalent interactions are between the nitrogen atom of the cyano and the adjacent hydrogen atom on the ring system.

7. The framework of claim 4, wherein the framework contains a plurality of pores from about 3 Å to about 5 Å.

8. The framework of claim 7, wherein the pores are from about 3.5 Å to about 4.5 Å.

9. The framework of claim 4, wherein framework has a surface area from about 300 m²/g to about 500 m²/g as measured by the Brunauer-Emmett-Teller method.

10. The framework of claim 10, wherein the surface area is from about 375 m²/g to about 425 m²/g.

11. The framework of claim 4, wherein the framework further comprises an alkene.

12. The framework of claim 11, wherein the alkene is ethylene.

13. A method of separating a C2-C6 alkene from a mixture comprising contacting the mixture with the framework of claim 4.

14. The method of claim 13, wherein the mixture comprises a mixture of C1-C6 alkane and C2-C6 alkene.

15. The method of claim 14, wherein the alkane is ethane.

16. The method of claim 13, wherein the alkene is ethylene.

17. The method of claim 13, wherein the method is carried out at a temperature below 100° C.

18. The method of claim 13, wherein the method is carried out at a temperature from about 40° C. to about 80° C.

19. The method of claim 13, wherein the framework has a selectivity for alkene over alkane of greater than 10.

20. The method of claim 13, wherein the method is carried out at a pressure from about 0.25 bar to about 5 bar.
